(19) United States
(12) Patent Application Publication
(10) Pub. No.: US 2005/0277134 A1
(43) Pub. Date: Dec. 15, 2005

(54) Method for amplifying nucleic acid and analysis of single-nucleotide polymorphism using the same

(76) Inventors: Kazunori Okano, Tokyo (JP); Yukie Nakashima, Shiki (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/072,510

(22) Filed: Mar. 7, 2005

(30) Foreign Application Priority Data
May 20, 2004 (JP) 2004-150172

(51) Int. Cl. C12Q 1/68; C12P 19/34

(52) U.S. Cl. 435/6; 435/91.2

(57) ABSTRACT

A method amplifies a nucleic acid by preparing an oligonucleotide being capable of complementarily hybridizing with a specific region of a target nucleic acid containing at least one mutation site, the oligonucleotide having at least one sequence being non-complementary to any of possible sequences of the at least one mutation site, subjecting the oligonucleotide to hybridization with the target nucleic acid, and carrying out a complementary-strand synthesis. A single-nucleotide polymorphism is analyzed using this method.

The present application claims priority from Japanese application JP 2004-150172 filed on May 20, 2004, the content of which is hereby incorporated by reference into this application.

### FIELD OF THE INVENTION

The present invention relates to a method for amplifying a nucleic acid, and the analysis of a single-nucleotide polymorphism using the method. More specifically, it relates to a method for amplifying a nucleic acid without being affected by a polymorphism at a priming site, a method for analyzing a single-nucleotide polymorphism using the method for amplifying a nucleic acid, and a kit for use in these methods.

### BACKGROUND OF THE INVENTION

The Human Genome Project for decoding the whole human genome completed April, 2003, and information on human genome nucleic acid sequences as a basic design of human being can now be utilized. Gene expressions and gene polymorphisms by genetic epidemiological approaches, both relating to diseases, will be increasingly researched. Such gene polymorphisms include, for example, single-nucleotide polymorphisms (SNPs), in which one base is replaced with another base; variable numbers of tandem repeat (VNTR), in which a repeating unit comprising several bases to several ten bases shows polymorphism; microsatellite polymorphisms, in which a repeating unit comprising two to four bases shows polymorphism; base deletion and base insertion.

Among them, SNPs occupy 90% or more of the entire gene polymorphisms, from which useful information on predisposition of diseases, drug resistance and/or drug efficacy upon administration may be obtained. SNPs occur at a high incidence, i.e., one per about 1,500 bases, show a small number of types of alleles and can be easily typed. The term “allele” means a base type of a specific gene, and refers to a sequence diversity at a specific sequence site in SNPs. Most of SNPs each have two different base sequences as the allele. A technique of analyzing a large number of different SNPs in a large number of different specimens at high speed is required to find effective SNPs, and various techniques for analyzing SNPs in a high throughput have been developed and used in practice.

The present inventors have established fundamentals of a bioluminometric assay coupled with modified primer extension reactions (BAMPER) (Guo-hua Zhou, et al., Nucleic Acid research, 29, e93 (2001)) and made further investigations on this technique for practical use as a technique suitable for analyzing effective SNP whose causal relation with diseases has been revealed in respective specimens, in a sponsored research by the New Energy and Industrial Technology Development Organization, a government-affiliated organization of Japan. BAMPER is a technique for analyzing a SNP using bioluminescence. More specifically, when two different probes corresponding to SNP alleles (base types) are subjected to extension, the extension proceeds only upon the use of a probe having a base sequence corresponding to the base type of the target SNP. BAMPER can determine the base type of a target SNP within several minutes by converting pyrophosphate generateed as a result of extension into ATP, carrying out a luciferin-luciferase reaction using the resulting ATP for light emission, and detecting and analyzing the light emission. This technique uses a simple reaction system to which reagents are to be added and enables analysis of a SNP using a luminescence measuring device having a simple optical system and can be suitably used in clinical sites and general laboratories.

Many of techniques for analyzing a SNP, including BAMPER, use an amplified product as a sample, which product has been amplified by a polymerase chain reaction (PCR) of a region containing a target SNP derived from a genomic DNA. The target SNP is typed by DNA sequencing using the PCR product, by carrying out a complementary strand extension (complementary-strand synthesis) using a primer (probe) being designed to have a sequence corresponding to the target SNP at the 3′ end and determining the presence or absence of a product of the complementary strand synthesis by electrophoresis (Japanese Unexamined Patent Application Publication No. 02-042999 (Japanese Patent No. 2853864)), or by analyzing a product of the complementary strand synthesis by bioluminescence as in BAMPER.

In such a PCR or complementary-strand synthesis using a genomic DNA or PCR product as a template, the amplification or the complementary-strand synthesis proceeds as a result of the complementary coupling of a primer or probe with a priming site containing a specific DNA sequence. An organism which proliferates in such a reproduction manner as to generate polymorphisms, namely non-parthenogenesis manner, has a pair of genomes having partially different alleles. The presence of the pair of genomes invites some problems. More specifically, if one of such sites having partially different sequences (90% of which is believed to be SNPs) is selected as a priming site, one of the pair of alleles which is entirely complementary to the primer (probe) is satisfactorily amplified, but the other allele partially different from the primer (probe) is amplified in a less amount. This is because a primer (probe) containing 17 to 28 bases generally shows a decreased efficiency in hybridization even if only one base is different. Even when the alleles are different in only one base, the genomic DNA show different holding properties of the priming site and show different (decreased) stability after hybridization with the primer (probe). Thus, one allele alone of the pair of genomes may be preferentially amplified under some reaction conditions. If one of the alleles of another SNP in the priming site is preferentially amplified, the alleles of the target SNP cannot be accurately determined.

As a possible solution to this problem, a region containing no SNP may be selected as a priming site. Such a priming site containing no SNP, however, is difficult to be set, because there are many limitation on the selection, such as self-holding and matching of melting temperatures of the pair of primers, and some of genes have a large number of SNPs. This technique of selecting a region containing no SNP as a priming site is not a general solution.

As another possible solution, an oligonucleotide as a primer is synthesized by using a mixture of two bases to be complementary to the two alleles, or two oligonucleotides being entirely complementary to the two alleles, respectively, are synthesized and are used as a mixture. In these cases, two primers having different sequences are competitively reacted. Such a competitive reaction, however, causes an error in determination or typing of a SNP basically by determining the ratio of amounts of alleles of the target SNP, because it is difficult to match thermodynamic parameters in hybridization. These problems reside not only in determination or typing of a SNP but also in general DNA polymerase reactions in which primers or probes are hybridized and an extension (complementary-strand synthesis) is carried out.

Japanese Unexamined Patent Application Publications No. 11-276179 and No. 10-75786 each disclose primer sequences containing an arbitrary base (N) for typing or classification. These techniques, however, use primer sequences in which a non-specified base is represented by an arbitrary base (N), and they are not intended to use a sequence being not complementary to any of possible sequences.

### SUMMARY OF THE INVENTION

Accordingly, an object of the present invention is to provide a method for analyzing a SNP, which is adaptable to a wider variety of applications, in which the amplification by PCR less varies depending on the difference in alleles even when a priming site includes one or more SNPs.

The present inventors have intended to avoid influence of one or more SNPs in a priming site by using not a primer hybridizing with both of two alleles in the SNPs in the priming site in a similar manner but a primer not hybridizing with the SNPs.

Specifically, the present invention provides, in an aspect, a method for amplifying a nucleic acid, including the steps of preparing an oligonucleotide being capable of complementarily hybridizing with a specific region of a target nucleic acid containing at least one mutation site, the oligonucleotide having at least one non-complementary sequence being not complementary to any of possible sequences of the at least one mutation site, subjecting the oligonucleotide to hybridization with the target nucleic acid, and carrying out a complementary-strand synthesis.

In the method, the oligonucleotide is allowed to hybridize with the target nucleic acid preferably at temperatures of 45° C. to 55° C. and more preferably at temperatures of 47° C. to 52° C.

The non-complementary sequence can be arranged at the third to fifteenth base from the 3′ end of the oligonucleotide.

The non-complementary sequence can be a base being not complementary to any of possible sequences of the at least one mutation site or can be a spacer not hybridizing with any base. The base includes, for example, naturally-occurring bases and modified bases.

A region of the oligonucleotide to hybridize with target nucleic acid may comprise generally about 17 bases or more, and preferably about 17 to about 28 bases in length.

The method for amplifying a nucleic acid sequence according to the present invention is useful for avoiding influence by other mutations present in the vicinity of the target single-nucleotide polymorphism, specifically in a region between the target single-nucleotide polymorphism and the 3′ end. Specifically, the present invention provides, in another aspect, a method for analyzing a single-nucleotide polymorphism, including the steps of preparing an amplified product by the method for amplifying a nucleic acid sequence of the present invention, and typing a single-nucleotide polymorphism in the target nucleic acid sequence other than the at least one mutation by the analysis of the amount of the amplified product. In this method, a base at the 3′ end or a second base from the 3′ end of the oligonucleotide is so designed as to correspond to the target single-nucleotide polymorphism.

The single-nucleotide polymorphism in the method for analyzing a single-nucleotide polymorphism may be typed by a process utilizing bioluminescence (BAMPER) and including the following steps of:
(1) converting pyrophosphate into ATP, the pyrophosphate being generateed as a result of the complementary-strand synthesis,
(2) carrying out a luminous reaction with the use of the resulting ATP and one or more enzymes, and
(3) analyzing the amounts of an amplified product based on the quantity of light emitted as a result of the luminous reaction to thereby type the single-nucleotide polymorphism.

In yet another aspect, the method for amplifying a nucleic acid sequence is used for preparing a sample for analyzing a single-nucleotide polymorphism and is useful in PCR amplification of a nucleic acid fragment containing the single-nucleotide polymorphism when the priming site includes one or more mutations other than the target single-nucleotide polymorphism. Specifically, the present invention further provides a method for analyzing a single-nucleotide polymorphism, including the steps of preparing an amplified product by the method for amplifying a nucleic acid of the present invention, and typing a single-nucleotide polymorphism in the target nucleic acid other than the at least one mutation with the use of the amplified product as a template.

The method just mentioned above may further include the steps of subjecting an oligonucleotide probe to hybridization with the amplified product, the oligonucleotide probe being so designed as to have a base corresponding to the single-nucleotide polymorphism site at the 3′ end or at a second base from the 3′ end, carrying out a complementary-strand synthesis to yield an amplified product, and typing the single-nucleotide polymorphism by the analysis of the amount of the amplified product. The single-nucleotide polymorphism can be typed, for example, by BAMPER.

The oligonucleotide probe may have at least one non-complementary sequence being not complementary with any of possible sequences of one or more mutation sites in a region of the target nucleic acid corresponding to the probe other than the single-nucleotide polymorphism. Using a probe having this configuration enables accurate typing of a single-nucleotide polymorphism by avoiding influence by other mutations, such as single-nucleotide polymorphisms, present in a set region for probe (priming site).

In addition and advantageously, the present invention provides a kit for use in the method for amplifying a nucleic acid and the methods for analyzing a single-nucleotide polymorphism. The kit includes an oligonucleotide primer or probe being capable of complementarily hybridizing with a specific region of a target nucleic acid containing at least one mutation site, wherein the oligonucleotide primer or probe has at least one non-complementary sequence being not complementary to any of possible sequences of the at least one mutation site.

The present invention also relates to a kit for analyzing a single-base polymorphism including an oligonucleotide probe containing an oligonucleotide capable of complementarily hybridizing with a specific region of a target nucleic acid containing at least one mutation site. In the kit, the oligonucleotide has at least one non-complementary sequence being not complementary to any of possible sequences of the at least one mutation site, and the oligonucleotide is so designed as to have a base at the 3′ end or a second base from the 3′ end corresponding to the single-nucleotide polymorphism site.

The present invention enables carrying out a complementary-strand synthesis by the action of a DNA polymerase without being affected by a mutation such as a single-nucleotide polymorphism in a priming site. This can avoid a phenomenon in which a nucleic acid fragment derived from one of a pair of alleles is preferentially amplified in a complementary-strand synthesis and a nucleic acid fragment derived from the other allele is amplified in a less amount. Accordingly, the target nucleic acid can be amplified while maintaining the ratio of amounts of nucleic acid fragments derived from the respective alleles. The method of the present invention enables accurate typing of a target SNP without being affected by other SNPs, if present, in the vicinity of the target SNP.

Further objects, features and advantages of the present invention will become apparent from the following description of the preferred embodiments with reference to the attached drawings.

### BRIEF DESCRIPTION OF THE DRAWINGS

FIG. 1 illustrates spacers for use in the present invention;

FIG. 2 shows the sequence of a region (part of AC 009563) in the vicinity of the target SNP according to Example 1;

FIG. 3 illustrates a PCR amplification according to a conventional method;

FIG. 4 illustrates a PCR amplification according to the present invention;

FIG. 5 shows a result in the PCR amplification according to a conventional method;

FIG. 6 shows a result in the PCR amplification according to the present invention;

FIG. 7 shows another result in the PCR amplification according to the present invention;

FIG. 8 shows results in determination of a SNP in 520 specimens according to the present invention;

FIG. 9 illustrates determination of a SNP by a complementary-strand synthesis; and

FIG. 10 shows results in determination of a SNP by a complementary-strand synthesis, in which the result 190-1 shows determination according to a conventional method, and the results 190-2, 190-3 and 190-4 show determination according to the present invention.

### DETAILED DESCRIPTION OF THE INVENTION

1. Method for Amplifying Nucleic Acid Sequence

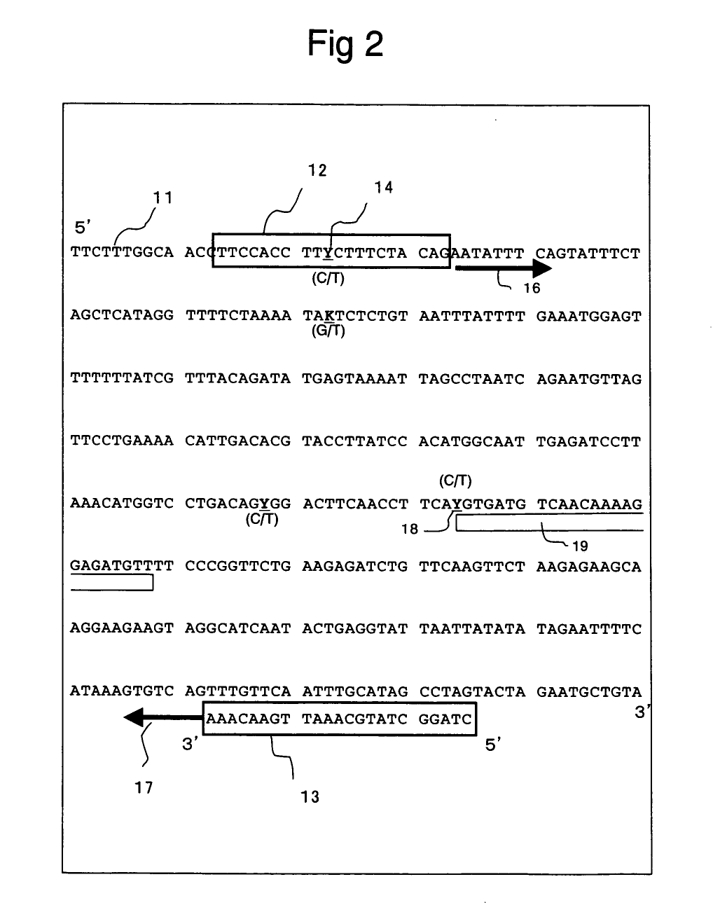

The method for amplifying a nucleic acid sequence of the present invention utilizes a nucleic acid synthesis reaction with the use of an oligonucleotide serving as a primer or probe. The oligonucleotide is capable of complementarily hybridizing with a specific region of a target nucleic acid containing at least one mutation site and has at least one non-complementary sequence being not complementary to any of possible sequences of the at least one mutation site. The term “to complementarily hybridize” has the same meaning as “to have a base sequence complementary to the specific region, except for a specific non-complementary sequence introduced according to the present invention”. The method of the present invention can amplify a template nucleic acid without being affected by one or more mutations such as single-nucleotide polymorphisms, if present, at a priming site (a set region for hybridization of the primer or probe). The advantages of the present invention will be illustrated with reference to FIGS. 2 to 4.

A sequence 11 in FIG. 2 is part of the Werner helicase gene. A genome region containing a target SNP 18 to be analyzed is subjected to PCR amplification using a primer 12 and a primer 13 in advance of typing of the single-nucleotide polymorphism. With reference to FIG. 2, a priming site of the template sequence with which the primer 13 is to hybridize includes no other SNP than the target SNP. A priming site of the genome sequence with which the primer 12 is to hybridize, however, includes another SNP 14 having alleles of Y, i.e., C and T, other than the target SNP.

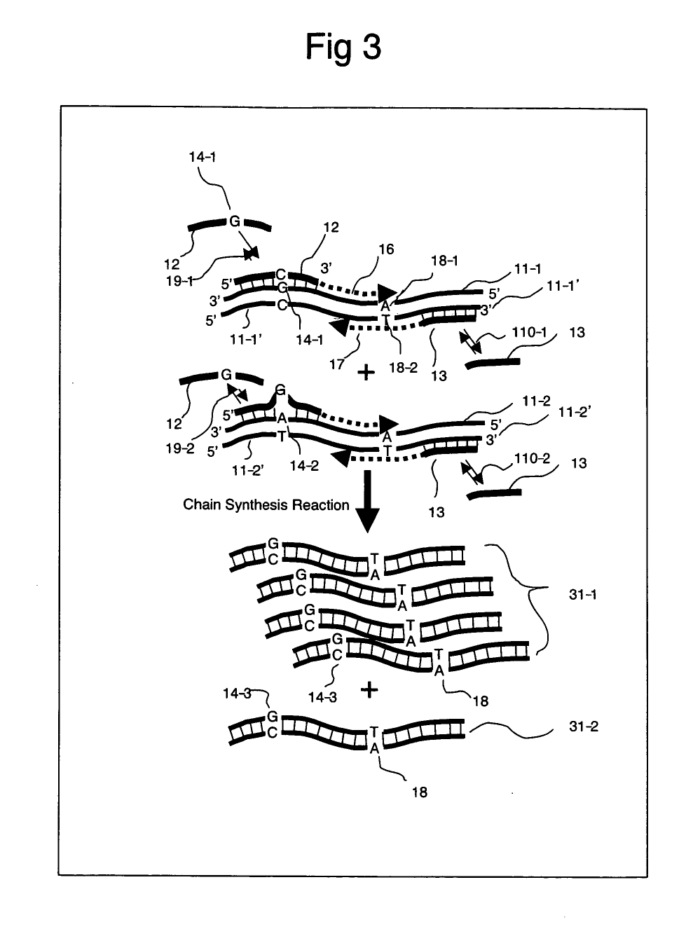

With reference to FIG. 3, when genomic DNA in which the SNP 14 is a C/T heterozygote are subjected to PCR amplification using regular primers 12 and 13, the primers 12 and 13 both hybridize with two pairs of genomic DNA (11-1 with 11-1′, and 11-2 with 11-2′) having different sequences at the SNP 14 site. The priming site with which the primer 13 hybridizes includes no SNP, and the primer 13 is combined with the genomic DNA 11-1′ and 11-2′ at an identical thermodynamic binding constant. A melting temperature Tm is generally employed as such a thermodynamic binding constant. However, the priming site with which the primer 12 hybridizes includes the SNP 14-1 and the SNP 14-2, and the primer 12 is non-complementary to the SNP 14-2. The primer 12 is combined with the genomic DNA 11-2 less thermodynamically stably than the genomic DNA 11-1, and the amount of a PCR product 31-2 of the allele having the SNP 14-2 is less than that of a PCR product 31-1 of the allele having the SNP 14-1.

This occurs only in a first reaction in PCR and does not occur in second and latter reactions in which the base at the priming site is replaced with another base 14-3 which is complementary to the primer sequence. It has been experimentally revealed that the amount of an amplified product of a non-complementary allele is generally 10% or less of that of an amplified product of a complementary allele when such a SNP is present within two bases from the 3′ end of the primer, whereas the amount varies depending on the sequence. In the case shown in FIG. 2, in which the SNP is present at the eleventh base from the 3′ end of the primer, the amount of an amplified product of a non-complementary allele decreases to about 60% of that of an entirely complementary allele, since the priming site has a sequence rich in AT. Although the amplification factor varies also significantly depending on the annealing temperature of the primer and/or salt concentration, the amount of an amplified product of a primer having a non-complementary base to a template DNA generally decreases due to the low thermodynamic stability, as compared with that of an amplified product of an entirely complementary primer.

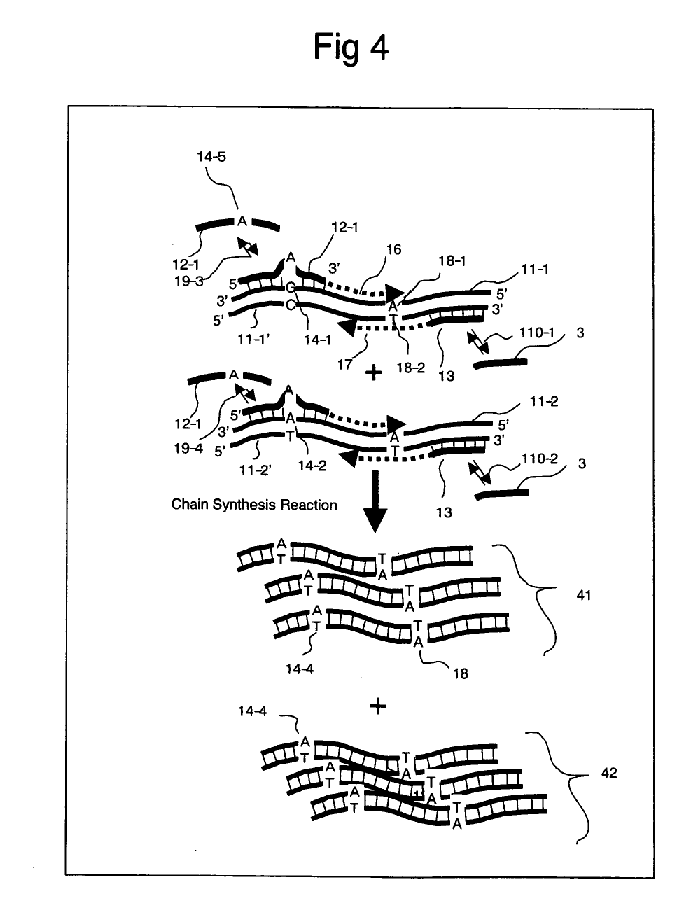

In contrast, according to the present invention, a nucleic acid synthesis reaction is carried out by using an oligonucleotide primer or probe 12-1 having a sequence being non-complementary to any of the sequences of the SNPs 14-1 and 14-2 in the priming site (FIG. 4). The primer 12-1 is non-complementary to the SNP 14-1 and the SNP 14-2 and is combined with them at an thermodynamically identical binding constant. This results in the substantially same amounts of the PCR products 41 and 42, since the polymerase chain reaction is not affected by the SNPs present in the priming site.

2. Analysis of Single-nucleotide Polymorphism (SNP)

The method for amplifying a nucleic acid sequence of the present invention is useful, for example, for avoiding influence of one or more SNPs other than a target SNP in a priming site in PCR amplification of a nucleic acid fragment containing the target SNP, which PCR amplification is carried out in advance of the analysis of the target SNP.

The method for amplifying a nucleic acid sequence of the present invention is also useful for avoiding influence by other SNPs in the vicinity of the target SNP, specifically those present between the 3′ end and the target SNP.

In addition, the method is useful for avoiding influence of one or more SNPs other than a target SNP in a priming site and avoiding influence by other SNPs in the vicinity of the target SNP, specifically those present between the 3′ end and the target SNP, in the PCR amplification of a nucleic acid fragment containing the target SNP, which PCR amplification is carried out in advance of the analysis of the target SNP.

3. Optimal Conditions for Complementary Strand Synthesis Reaction

Optimal conditions for a complementary-strand synthesis in the method for amplifying a nucleic acid or the method for analyzing a single-nucleotide polymorphism according to the present invention will be described.

(1) Annealing Temperature

The oligonucleotide (primer or probe) for use in the present invention has at least one non-complementary sequence (mismatch) to a template nucleic acid and may have a decreased melting temperature Tm. There is a potential for the amount of a PCR product to decrease. To avoid this, the hybridization between the template nucleic acid and the oligonucleotide (primer or probe) is preferably carried out at temperatures lower than that in regular procedures, i.e., at 45° C. to 55° C., and more preferably at 47° C. to 52° C. in the method for amplifying a nucleic acid of the present invention.

(2) Base Length of Oligonucleotide

Introducing one or more sequences (mismatches) non-complementary to the template into the oligonucleotide (primer or probe) reduces the melting temperature Tm of the oligonucleotide (primer or probe). An excessively large amount of introduced mismatches may reduce the efficiency of hybridization with the template. The number of mismatches to be introduced is preferably one per about ten bases. A primer comprising 28 bases in length is preferably incorporated with about two or less mismatches. If a large number of SNPs is present in the vicinity of the priming site and a large number of mismatches must be introduced, the primer preferably further comprises one to three additional bases per one more mismatch at the 5′ end side, with respect to the base length of an entirely complementary primer or probe.

When the oligonucleotide has about one to two mismatches, the base length of a region of the oligonucleotide to hybridize with the template nucleic acid is generally 17 bases or more, and preferably 17 to 28 bases in length. The phrase “the base length of a region of the oligonucleotide to hybridize with the template nucleic acid” means that, if the oligonucleotide primer or probe includes one or more sequences not hybridizing with the template nucleic acid sequence, the one or more sequences are not included in the base length. For example, in an F1 primer (F1c+F2) in loop-mediated isothermal amplification (LAMP) having an F1c sequence being complementary to the template and an added F2 sequence being non-complementary to the template, the F1 sequence preferably comprises 17 to 28 bases in length.

(3) Position of Introduced Non-complementary Sequence (Mismatch)

The at least one mismatch can be introduced at any position and is preferably introduced at the third to fifteenth base from the 3′ end of the primer or probe for better advantages of the present invention.

(4) Type of Non-complementary Sequence (Mismatch)

The mismatch to be introduced into the oligonucleotide (primer or probe) for use in the present invention is not specifically limited, as long as it has such a structure as not to hybridize with any of possible sequences of the mutation site in the template nucleic acid. Among four bases in the genomic DNA, a base being non-complementary to any of the sequences (bases) of the mutation site can be used as the mismatch. If a SNP in the priming site is of A/G alleles, T or C can be used as the mismatch. The base as the mismatch may be a modified base.

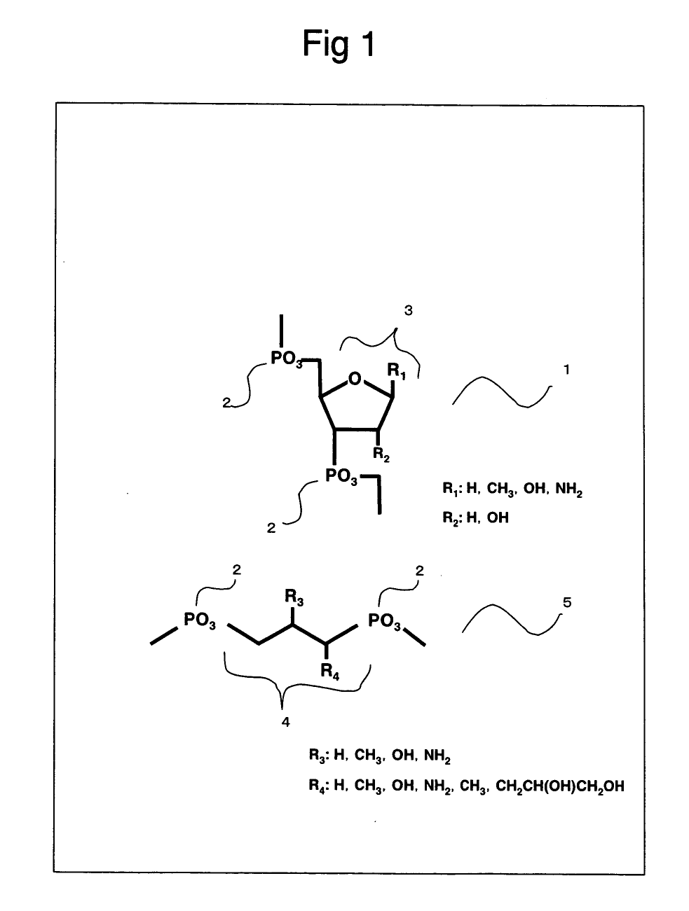

Alternatively, a structure which does not form an effective hydrogen bond with the sequence (bases) of the mutation site can be used as a spacer. The “spacer” for use in the present invention means a structure which does not form an effective hydrogen bond with the corresponding template sequence (bases) and simply serves to connect between adjacent bases. The spacer can be one sequence and can be either of a naturally occurring substance or non-naturally occurring substance. Specific examples thereof are a structure 5 including a phosphodiester bond with the interposition of a glycelic group having no side chain, and a structure 1 including a phosphodiester bond with the interposition of ribose or 2-deoxyribose having no base, as shown in FIG. 1.

(5) Complementary Strand Synthesis Reaction

The reaction of complementary-strand synthesis according to the present invention is not limited to PCR and can be any nucleic acid synthesis reaction such as isothermal and chimeric primer-initiated amplification of nucleic acids (ICAN), loop-mediated isothermal amplification (LAMP), ligase chain reaction (LCR), nucleic acid sequence-based amplification (NASBA) or allele-specific polymerase chain reaction (ASP-PCR). The principle of the present invention for avoiding influence of mutations other than the target SNP by introducing one or more mismatches can also be applied to, for example, ICAN using an RNA/DNA chimera primer. The same advantages can also be obtained in LAMP by applying the principle of the present invention to the structure of the F1c region of the F1 primer which hybridizes with the template nucleic acid, as described above. The principle of the present invention can also be applied to introduction of a promoter region in NASBA.

(6) Typing of Single-nucleotide Polymorphism (SNP)

A target SNP can be typed by determining the amount of an amplified product by electrophoresis or by BAMPER utilizing bioluminescence. The amplified product herein is a product of a complementary strand synthesis using a probe being designed so as to have a base at the 3′ end or a second base from the 3′ end corresponding to target SNP. Details of the typing can be found in the following examples and the above-mentioned document (Guo-hua Zhou, et al., Nucleic Acid research, 29, e93 (2001)).

### EXAMPLES

The present invention will be illustrated in further detail with reference to several examples below, which are never intended to limit the scope of the invention.

Device and Reagent:

Devices and reagents used in the following examples are as follows. The temperature of extension was controlled by a thermal cycler DNA Engine Tetrad (MJ RESEARCH). The PCR products were analyzed by using a microchip electrophoresis analyzing system SV 1210 (Hitachi High-Technologies Corporation). The syntheses of oligonucleotides were entrusted to Sigma-Genosys. The DNA polymerase was obtained from Amersham Biosciences, and other reagents were general commercially available products. The genomic DNA was purified from the blood provided by volunteers. The test procedure was in accordance with Molecular Cloning (Cold Spring Harbor Laboratory Press, Molecular Cloning (Second edition), 1989), unless otherwise specified.

PCR Condition:

A polymerase chain reaction was carried out in the following manner. Each 1 μL of a 10 zmol/μL (1×10⁻²⁰ mol/μL) genomic DNA sample was placed in a 96-well PCR plate, and the plate was placed on ice. To each well were added 0.2 μL of 2.5 unit/μL Taq. DNA polymerase (QIAGEN), 4 μL of 2.5 mM dNTPs, and 0.8 μL each of a set of 25 pmol/μL primers. The volume was adjusted with sterile water to 100 μL per each well. The volumes of components can be changed while maintaining the same proportions. For example, PCR can be carried out on a 50 μL scale. The plate was sealed with a pressure-sensitive adhesive sheet and was placed in a thermal cycler. After heating at 94° C. for two minutes for degenerating the genome, a thermal cycle of 94° C. for 30 seconds, 57° C. for 30 seconds, and 72° C. for 1 minute was repeated a total of 35 to 40 times.

Electrophoretic Analysis of PCR Product:

The amount of the PCR product was determined by a microchip electrophoresis analyzing system SV 1210 (Hitachi High-Technologies Corporation) using an i-SDNA12 kit (Hitachi High-Technologies Corporation) as a reagent kit. The system can automatically determine the length and amount of the target PCR product based on the length and amount of a base as an internal standard marker within an analysis range of 10 to 500 base pairs (bp). A total of 1 μL of the polymerase chain reaction mixture was analyzed in each example according to the manual.

### Example 1

In this example, the present invention is applied to typing of a target single-nucleotide polymorphism (SNP), in which a region in the vicinity of the target SNP is amplified by PCR and the priming site contains another SNP in addition to the target SNP, as illustrated in FIGS. 2 to 8.

A sequence 11 (SEQ ID NO: 1) shown in FIG. 2 is a partial sequence of a template strand containing a target SNP 18, and information on the sequence containing this region is available as Accession Number AC 009563 (the Werner helicase gene) from the database of NCBI (http://www.ncbi.nlm.nih.gov/). In FIG. 2, the sequence of a sense strand is indicated in a direction from the 5′ end to the 3′ end. Initially, a genome sequence region containing the target SNP 18 was amplified by PCR using a primer 12 (SEQ ID NO: 2) and a primer 13 (SEQ ID NO: 3) to thereby form samples for typing of the target SNP 18. The priming site corresponding to the sequence of the primer 12, however, includes another SNP 14 than the target SNP 18. The base type of the SNP 14 is Y including two alleles of base C and base T. Thus, there are three possible gene types on the SNP site including a T/T homozygote, a C/C homozygote and a T/C heterozygote.

(1) Amplification of SNP Region Using Regular Primers

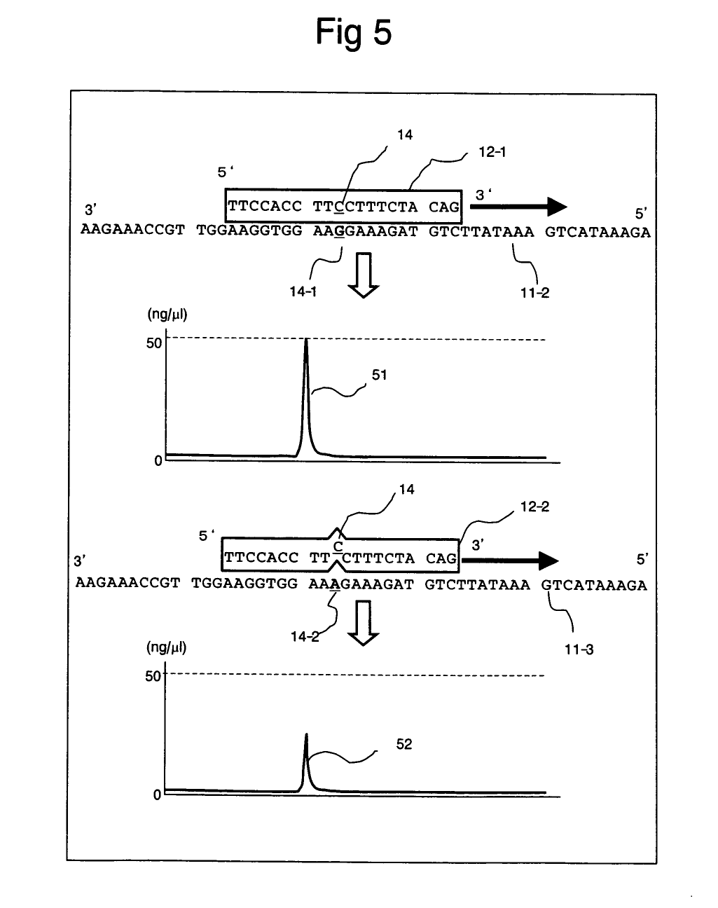

Initially, PCR was carried out by using the primers 12 and 13 corresponding to the allele having base C corresponding to the SNP 14 of the template (FIGS. 3 and 5). Human genomic DNA which had been verified to have base C (G in a complementary strand) or T (A in a complementary strand) were used as the template, and the PCR was performed under the above condition.

Two sequences 11-2 and 11-3 shown in FIG. 5 are part of complementary strands (antisense strands) to the sequence 11 in the genes having base C or base T as the SNP 14. The complementary strand sequence 11-2 has G as the sequence 14-1 corresponding to the SNP 14 and entirely complementarily hybridizes with the primer 12 (a hybrid 12-1 in FIG. 5). The complementary strand sequence 11-3, however, has A as the sequence 14-1 corresponding to the SNP 14, and the primer 12 is non-complementary to the template at the SNP site 14-2 (a hybrid 12-2 in FIG. 5). The hybrid 12-2 has thermodynamic stability lower than that of the hybrid 12-1. The resulting PCR products were analyzed by electrophoresis to find that the peak 52 of the product of a sample as the hybrid 12-2 is about one half of a peak 51 of a product of a sample as the entirely complementary hybrid 12-1.

The target SNP 18 cannot be accurately analyzed by using such PCR products as a template. The case where the SNP 14 is a T/C heterozygote will be taken as an example, with reference to FIG. 3. With reference to FIG. 3, one of the alleles (11-1 and 11-1′) has base C (G in the complementary strand) as the SNP 14 (14-1) and base T (A in the complementary strand) as the target SNP 18. The other of the alleles (11-2 and 11-2′) has base T (A in the complementary strand) as the SNP 14 (14-2) and base T (A in the complementary strand) as the target SNP 18. When a polymerase chain reaction is carried out on this genomic DNA using the primers 12 and 13, one of the alleles having base C as the SNP 14 yields an amplified product in a larger amount than that in the other allele, as shown in FIG. 5. A pair of genomic DNAs is not separately amplified in a polymerase chain reaction, and an amplified product derived from the two genomic DNAs cannot be distinguished and separated. Accordingly, the target SNP 18 is analyzed while one of the alleles is preferentially amplified.

(2) Amplification of SNP Region Using Primer According to the Present Invention

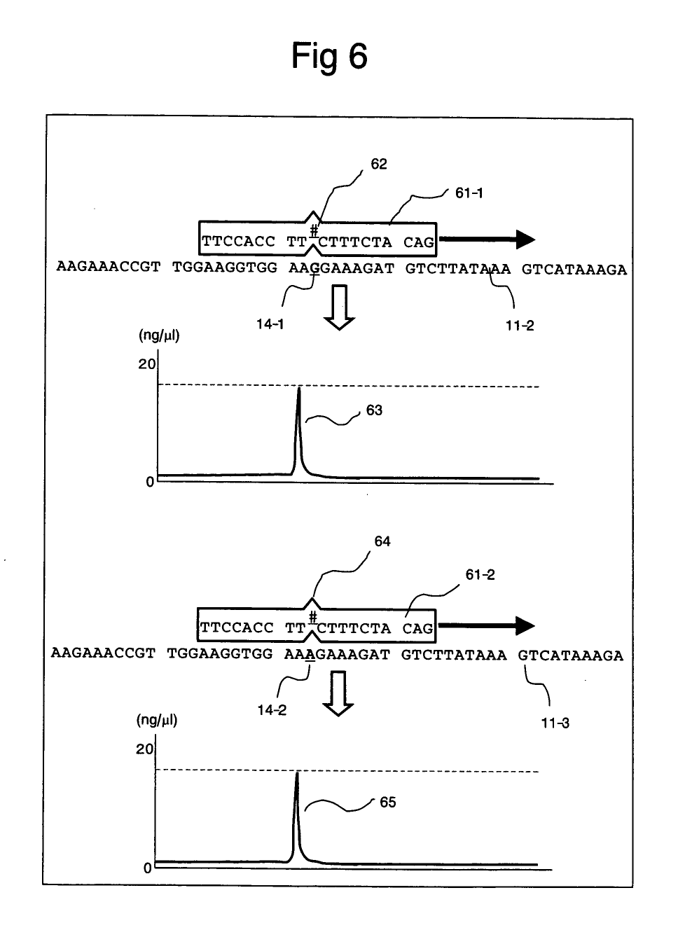

Next, a genomic DNA having the SNP 14 as a T/C heterozygote was amplified by a polymerase chain reaction using the primer according to the present invention (FIGS. 4 and 6).

A primer 12-1 has a base A (adenine) (14-5) which is non-complementary to any of the SNP sites 14-1 and 14-2 in the target genome (FIG. 4). The base 14-5 is not specifically limited, as long as it does not hybridize with any of the SNP sites 14-1 and 14-2 in the genomic DNA, and can be a spacer as shown in FIG. 1 instead of the base A. The primer according to the present invention can hybridize with the target genomic DNA in a thermodynamically constant amount, even when the priming site has one or more SNPs other than the target SNP. This can amplify any of the alleles in a constant proportion.

FIG. 6 illustrates a PCR amplification using primers 61-1 and 61-2 (both SEQ ID NO: 4) according to the present invention. The primers 61-1 and 61-2 each comprise the structure 1, wherein R₁ and R₂ are hydrogen, shown in FIG. 1 as a spacer represented by the symbol # at a sequence 62 corresponding to the SNP site 14-1. The primers 61-1 and 61-2 are non-complementary to any of the SNPs 14-1 and 14-2 of the templates, hybridize with any of the templates with relatively low but thermodynamically equivalent stability. The resulting PCR products upon use of the primers 61-1 and 61-2 according to the present invention were analyzed by electrophoresis to find that the primers 61-1 and 61-2 yield substantially identical product peaks 63 and 65. Namely, the polymerase chain reaction using the primers according to the present invention can amplify alleles having different bases at a SNP site, even if one or more SNPs are present in the priming site.

(3) Control of Annealing Temperature

In general, insertion of a non-complementary sequence into a primer invites a decreased annealing temperature and a decreased amount of a PCR product. This problem can be solved by carrying out annealing of a primer and a template strand at lower temperatures. In fact, the polymerase chain reactions shown in FIGS. 5 and 6 were carried out at an annealing temperature of 55° C., and the PCR product peaks 63 and 65 in FIG. 6 separated by electrophoresis are at substantially the same level as the amplified product peak 52 in FIG. 5, wherein the amount of the amplified product is less than the other one.

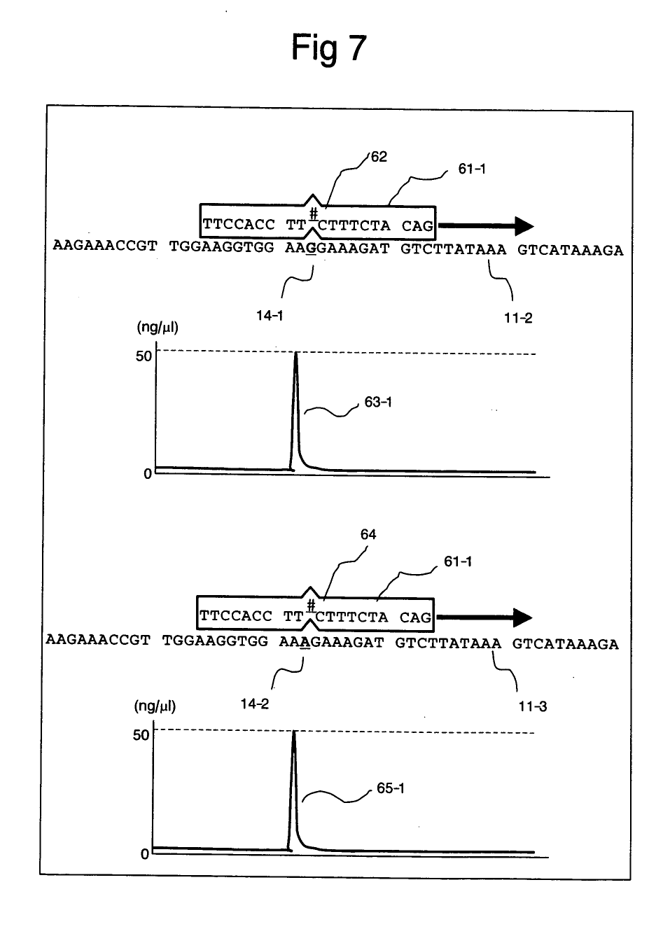

FIG. 7 illustrates polymerase chain reactions at an annealing temperature of 50° C. using primers according to the present invention. The other conditions than the annealing temperature are the same as in the polymerase chain reaction illustrated in FIG. 6. The resulting PCR product peaks 63-1 and 65-1 in FIG. 7 show that PCR products can be obtained in larger amounts by decreasing the annealing temperature by 5° C.

Specifically, these results show that a PCR can be carried out while maintaining the amount of an amplified product without being affected by SNPs, if any, in the priming site by using a primer having, as a base in the primer sequence corresponding to the SNP, a base or spacer being non-complementary to and not hybridizing with the base at the SNP site and by setting the annealing temperature at a temperature about 5° C. to about 10° C. lower than a regular annealing temperature.

(4) SNP Typing

The target SNP 18 was analyzed (typed) using the PCR products obtained in (1) and (2). Initially, the PCR products were purified by gel filtration with a Sephadex G100 (or ultrafiltration) for removing unreacted primers and dNTPs used in the polymerase chain reactions. The PCR products can be purified by enzymatic cleaning up in the following manner. Specifically, 0.7 μL of 1 unit/μL shrimp alkaline phosphatase, 0.06 μL of 10 unit/μL exonuclease I, 0.3 μL 10×PCR buffer (Amersham Pharmacia) and 3.94 μL of sterile water are added to 50 μL of the reaction mixture after the polymerase chain reaction. The mixture is subjected to an enzymatic reaction with incubation at 37° C. for 40 minutes, followed by inactivation of the enzyme by heating at 80° C. for 15 minutes. Gel filtration using Sephadex G25 or G50 or ultrafiltration after the enzymatic cleaning up can further purify the target PCR product to a higher purity.

Each of the purified PCR products was subjected to hybridization with probes 19 for SNP typing (SEQ ID NO: 5), followed by complementary strand extension. The probes 19 are so designed as to have the 3′ end at a position corresponding to the target SNP 18 (FIG. 2). A complementary-strand synthesis by the action of a DNA polymerase occurs when the 3′ end of a probe is complementary to the sequence of the target SNP 18 in the DNA strand 11. If not, the complementary-strand synthesis does not occur or occurs only to a small extent. When the base of the SNP 18 is C, for example, the complementary strand synthesis occurs only when the 3′ end of the probe 19 is G, and hardly occurs when the 3′ end of the probe 19 is A. In contrast, when the base of the SNP 18 is T, the complementary strand synthesis occurs only when the 3′ end of the probe 19 is A, and hardly occurs when the 3′ end of the probe 19 is G. Thus, the target SNP 18 can be typed by determining whether or not extension occurs upon use of probes having A and G at the 3′ end, respectively.

The extension can also be detected by converting pyrophosphate generateed during the complementary strand synthesis into ATP by the action of an enzyme (pyruvate orthophosphate dikinase) and determining the amount of ATP using a luciferin-luciferase system, for example, in the following manner. The reaction mixture of the polymerase chain reaction is subjected to enzymatic cleaning up in the same way as above and is cooled to 4° C. Each 2 μL of the reaction mixture is dispended into each well of a white 96-well PCR plate. A total of 1 μL of the probe 19 (5 pmoL/L) is added thereto. Separately, 0.0275 μL of 5 unit/μL Taq. DNA polymerase and 0.04 μL of 5 mM dNTPs are added to sterile water up to 1.0 μL. Each 1.0 μL of the resulting solution is added to each well. A mineral oil (4 μL) is placed thereon. A cycle of 94° C. for 10 seconds and 55° C. for 10 seconds is repeated a total of five times, followed by cooling to 25° C. Each 10 μL of a light-emitting reagent previously held to 25° C. is added, the resulting mixture is stirred by pipetting, and the light emission is determined using a luminometer. The light-emitting reagent is a bioluminescence kit (Kikkoman Corporation) for converting pyrophosphate into ATP and detecting ATP by the action of a luciferin and a luciferase. This procedure easily detects whether or not the extension occurs based on the emission intensity depending on the amount of pyrophosphate.

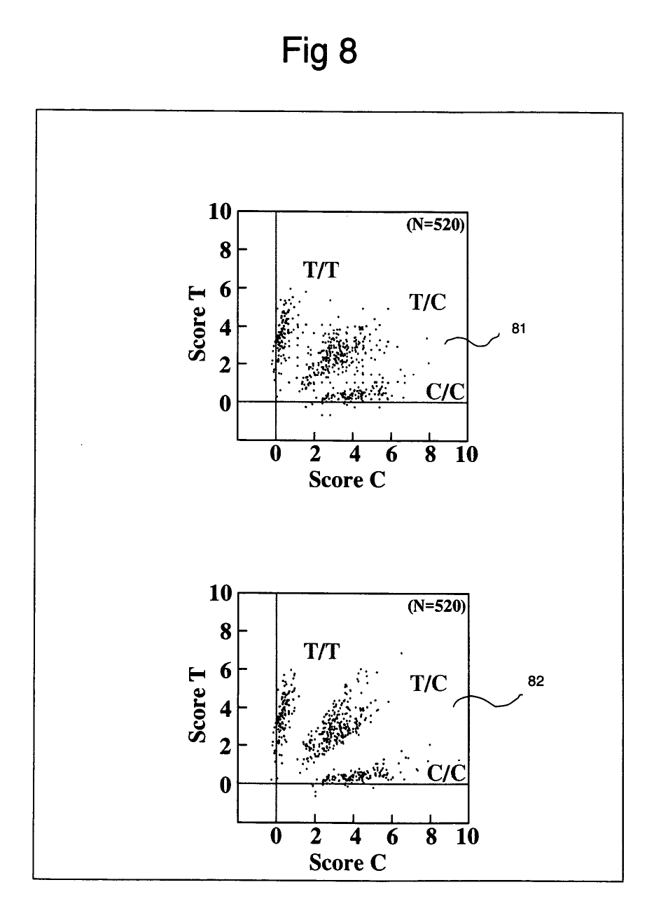

FIG. 8 shows an analyzed result 81 of the SNP 18 using, as templates, PCR products obtained upon use of the regular primers 12-1 and 13, and an analyzed result 82 of the SNP 18 using, as templates, PCR products obtained upon use of a primer 61-1 or 61-2 according to the present invention and the regular primer 13. The primers 61-1 and 61-2 have a non-complementary spacer 62 as bases in the site corresponding to possible SNPs 14-1 and 14-2. When the regular primers 12-1 and 13 are used, a group of T/C heterozygote shows a large variance, and the boundaries with a group of T/T homozygote and with a group of C/C homozygote are not clear, since the amount of the amplified product varies depending on the type of the base at the SNP 14 in the template alleles (the result 81 in FIG. 8). In contrast, the groups of T/C heterozygote, T/T homozygote and C/C homozygote, respectively, show clear boundaries with each other, and the determination precision is 99.7% or more (6σ or more) when a PCR using the primer according to the present invention is carried out and the amplified products thereof are used as samples (the result 82 in FIG. 8).

In this example, amplification by PCR is taken as an example, but the present invention can also be applied to any amplification in which an oligonucleotide primer or probe complementary to a template is hybridized at a priming site, and a complementary-strand synthesis is carried out, as in nucleic acid sequence-based amplification (NASBA) or rolling-circle amplification.

### Example 2

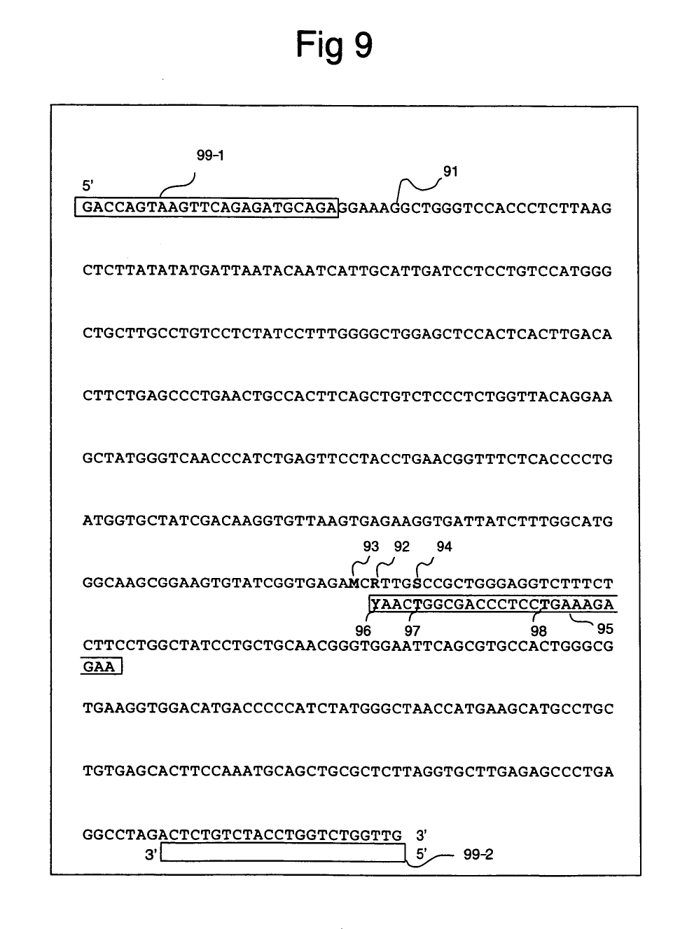

In this example, the present invention is applied to complementary strand extension for use in typing of a specific single-nucleotide polymorphism, in which a template DNA to hybridize with a probe has one or more other SNPs in addition to the target SNP, as shown in FIG. 9.

A sequence 91 (SEQ ID NO: 6) of the CYP1A1 gene shown in FIG. 9 is a partial sequence of a template strand including a target SNP 92. The information on the sequence containing this region is available as Accession Number X02612 from the database of NCBI (http://www.ncbi.nlm.nih.gov/). In FIG. 9, the sequence of a sense strand is indicated in a direction from the 5′ end to the 3′ end. The base of the target SNP 92 is Y, i.e., there are two alleles including base A and base G. Other SNPs 93 and 94 are present in the vicinity of the target SNP 92. The base of the SNP 93 is M, i.e., there are two alleles including base A and base C. The base of the SNP 94 is S, i.e., there are two alleles including base G and base C.

Initially, a PCR was carried out in the same way as in Example 1, except for using a pair of primers 99-1 (SEQ ID NO: 7) and 99-2 (SEQ ID NO: 8) being designed so as to sandwich the target SNP 92, to thereby amplify the genome sequence region 91 including the target SNP 92. The PCR products were purified by enzymatic cleaning up by the procedure of Example 1. The purified PCR products were subjected to hybridization with a probe 95 (SEQ ID NO: 9) for SNP determination, followed by complementary strand extension. The 3′ end 96 of the probe 95 corresponds to the position of the target SNP 92. As the probe 95, two probes having base C and base T, respectively at the 3′ end 96 were prepared.

A complementary-strand synthesis by the action of a DNA polymerase occurs when the 3′ end of the probe 95 is complementary to the sequence of the target SNP 92 in the DNA strand 91. If not, the complementary-strand synthesis does not occur or occurs only to a small extent. The target SNP 92 can be typed by determining whether or not extension occurs with the separate use of two probes having C and T, respectively, at the 3′ end.

Whether or not extension occurs was determined by the procedure of Example 1, by converting pyrophosphate generateed during the complementary strand synthesis into ATP, and determining the amount of ATP by using a luciferin-luciferase system. The presence of the other SNP 94 (G or C), however, in the template DNA to which the probes hybridize invites the following problem.

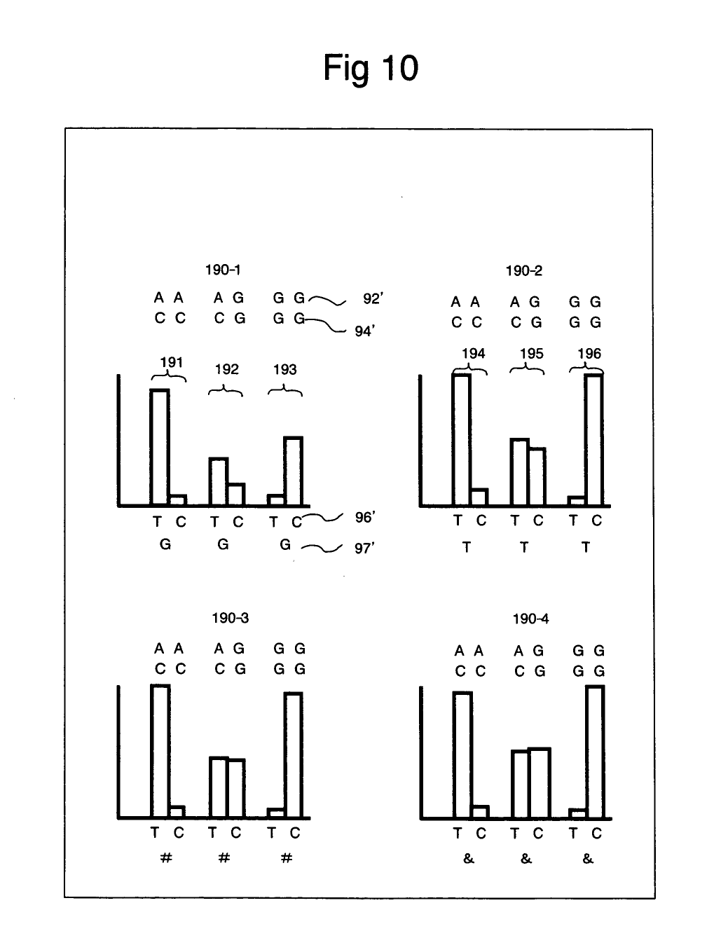

The SNP 94 is positioned four bases downstream from the target SNP 92 and is positioned at the fifth base from the 3′ end of the probe (FIG. 9). When a probe sequence 97 corresponding to the SNP 94 is non-complementary to the SNP 94, such as in the case where the SNP 94 is G and the corresponding probe sequence 97 is G, the amount of extension product of the probe decreases to about 50% to about 70% of that in the case where the two are entirely complementary, due to non-complementary property of the probe sequence 97, even when the 3′ end 96 of the primer is complementary to the target SNP 92 (FIG. 10).

The amount of the extension product of the probe decreases to 10% or less of that where the two are entirely complementary, when the probe sequence 97 corresponding to the SNP 94 is non-complementary to the SNP 94, such as in the case where the SNP 94 is G and the corresponding probe sequence 97 is G, and when the 3′ end 96 of the primer is non-complementary to the target SNP 92.

In contrast, the signal intensity is 100% when the probe sequence 97 corresponding to the SNP 94 is complementary to the SNP 94, such as in the case where the SNP is C and the corresponding sequence 97 is G, and the 3′ end 96 of the probe is complementary to the target SNP 92.

The amount of the extension product of the probe, however, decreases to 10% or less of that in the case where the two are entirely complementary, when the probe sequence 97 corresponding to the SNP 94 is complementary to the SNP 94, such as in the case where the SNP is C and the corresponding sequence 97 is G, but the 3′ end 96 of the probe is non-complementary to the target SNP 92.

In the case where the probe sequence 97 corresponding to the SNP 94 is non-complementary to the SNP 94, the SNP 96 which is complementary to the probe and is a homozygote may be misidentified as a heterozygote. In addition, when the SNP is a heterozygote, it may be misidentified as a homozygote, due to a lower signal intensity of one of the two alleles.

The actual frequency of gene polymorphism in the gene in question is unknown, but SNPs present in the vicinity of each other may often be linked. More specifically, the probe sequence 97 corresponding to the SNP 94, assuming that being a major allele, is non-complementary to a minor allele. One of the alleles relating to the SNP 96 behaves in the same manner, and thereby one of the alleles may not be apparently detected.

The probes according to the present invention can serve to determine the polymorphism of the target SNP in any of the above-mentioned cases. This will be described with reference to FIGS. 9 and 10. The SNP 94 is frequently C when the target SNP 92 in the genome sequence shown in FIG. 9 is A, and the sequence 97 is frequently G when the target SNP 92 is G.

It is assumed, for example, that the SNP 94 is frequently C when the target SNP 92 in the genome sequence shown in FIG. 9 is A, and the sequence 94 is frequently G when the target SNP 92 is G. In FIG. 10, the uppermost abscissa represents a possible allele 92′ (A/A homozygote, A/G heterozygote and G/G homozygote in this order from the left-hand) of the target SNP 92, and the second abscissa represents a possible allele 94′ (C/C homozygote, C/G heterozygote and G/G homozygote in this order from the left-hand) of the SNP 94 linked with the target SNP 92. The sequences 96′ and 97′ are sequences of the 3′ ends of the probes used in the respective polymorphisms. The ordinate represents a signal intensity of emitted light. Regular probes each having base G as the sequence 97 being complementary to the major allele of the SNP 94 were prepared. The signal intensity tends to decrease when the sequence 97 of the probe is G and the SNP 94 in the genome is G, as described above. The target SNP 92 was then analyzed by using the above-prepared probes having base T and base C at the 3′ end 96, respectively, corresponding to base A and base G of the sequence of the target SNP 92. The graph 190-1 in FIG. 10 illustrates a sequence 193 of the 3′ end of the probe. As a result, a sample 192 which is originally an A/G heterozygote shows a lower signal intensity with respect to the base type C. This is because the sequence of the SNP 94 is G when the sequence 92 (191) is G, but the probe used has G alone as the sequence 97 and is non-complementary to the template at this position, which inhibits sufficient extension of the probe. To avoid this, were used the probes according to the present invention each comprise a synthetic oligonucleotide having a base non-complementary to any possible sequences of single-nucleotide polymorphisms in the template polynucleotide. As an example of the probes according to the present invention, a probe having neither C nor G but T as the sequence 97 corresponding to the SNP 94 in the template was used. The results is shown as the graph 190-2 in FIG. 10, indicating that the probe can yield accurate results in all the samples 194 as an A/A homozygote, 195 as an A/G heterozygote, and 196 as a G/G homozygote. Separately, probes having a spacer of the structure 1 (herein represented by the symbol #, wherein R₁ and R₂ are hydrogens) or a spacer of the structure 5 (herein represented by the symbol &, wherein R₃ and R₄ are hydrogens) shown in FIG. 1 as the region 97 corresponding to the SNP 94 were used, and the results are shown in the graphs 190-3 and 190-4, respectively. The results show that the probes according to the present invention can yield accurate results.

The present invention advantageously enables an accurate determination of a target SNP even if the sequence region of the probe overlaps other SNP sites than the target SNP in the template and even if the overlapped region is in the vicinity of the 3′ end. It also enables arbitrary designing of probes with respect to a sequence having single-nucleotide polymorphisms at a known specific position and enables designing of probes without considering the difference in stability of probe hybridization due to the single-nucleotide polymorphism in question when a complementary-strand synthesis using a polymerase is carried out. The two probes can have substantially the same reactivities or reaction efficiencies by setting the annealing temperature about 5° C. to about 10° C. lower than a regular annealing temperature, as in Example 1.

### Example 3

Fluorescence-labeled probes were used for detecting the target SNP 92 in the DNA fragment 91 which had been amplified by PCR using the primers 99-1 and 99-2 according to Example 2. The design, hybridization conditions and extension conditions for the probes are the same as in the probes 95, except that the probes herein were labeled with two different fluorophores at the 5′ end so as to correspond to two possible bases of SNPs at the 3′ end. More specifically, one of the probes 95 had been labeled with Cy3 at the 5′ end and corresponded to base A as the target SNP 92. The Cy3 has an emission wavelength of 570 nm. The other of the probes 95 had been labeled with Cy5 at the 5′ end and corresponded to base G as the target SNP 92. The Cy5 has an emission wavelength of 649 nm. These probes were subjected to hybridization with the PCR products, and the wavelengths of emitted light were determined to thereby determine the type of the target SNP. In any case, an accurate result was obtained. In this example, the target SNP was typed by detecting light emission from two fluolophre having different emission wavelengths simultaneously. The typing, however, can also be carried out by using one fluolophre and detecting light emission using probes separately. A suitable fluolophre can be arbitrarily selected from among a variety of commercially available fluolophre according to the properties of a measuring apparatus to be used. The two probes can have substantially the same reactivities or reaction efficiencies by setting the annealing temperature about 5° C. to about 10° C. lower than a regular annealing temperature, as in Example 1.

### Example 4

The primer 99-1 and the probes 95 used in Example 2 were used in combination for PCR. The two different probes 95 were used for detecting the target SNP 92 as in Example 2. Only when the 3′ end 96 of the probe entirely matches with the target SNP 92, amplification occurs. Thus, the target SNP could be easily typed by detecting the presence or absence of an amplification product by electrophoresis. The two probes can have substantially the same reactivities or reaction efficiencies by setting the annealing temperature about 5° C. to about 10° C. lower than a regular annealing temperature, as in Example 1.

### Example 5

A micro array comprising fluorescence-labeled probes immobilized to a solid phase was used. The fluorescence-labeled probes were those used in Example 3, except for having base T instead of base A at the position 98. Thus, the higher order structure of the probes was destroyed, in order to carry out hybridization at relatively low temperatures. The genome sequence 91 was used as a sample, and the resulting signal intensities in all the combinations of the sequences of the target SNP 92 and the SNP 94 were compared. Hybridization was carried out at a temperature of 50° C. using 0.2M NaCl 10 mM Tris-HCl buffer at pH 7.5. The actual frequency of gene polymorphism in the gene used is unknown as in Example 2, but SNPs positioned in the vicinity of each other may often be linked. More specifically, the probe sequence 97 corresponding to the SNP 94, assuming that being a major allele, is non-complementary to a minor allele. In this case, one of the alleles relating to the SNP 96 behaves in the same manner as the linked allele, and thereby one of the alleles may not be apparently detected. This problem, however, can be avoided by using the probes according to the present invention.

When regular (conventional) probes were used, there are one case where two bases are entirely complementary, two cases where one base is non-complementary, and one case where two bases are non-complementary in the sequences (bases) of the target SNP 92 and the SNP 94, and the resulting signal intensities decreases in this order. The case where the two bases are non-complementary showed a decreased signal intensity of about 40% of that in the case where the two bases are complementary. In contrast, when the probes according to the present invention were used, the variation in signal intensity was within an error range of 8% relatively, showing that the present invention can avoid influence by other SNPs positioned in the vicinity of the target SNP in typing of the target SNP.

The method of the present invention can carry out a complementary-strand synthesis (nucleic acid amplification) without being affected by one or more mutations in a target nucleic acid. The present invention is therefore advantageously used for nucleic acid fragment amplification when one or more SNPs are present in a priming site of the target nucleic acid and for typing of a target SNP when one or more other SNPs are present in the vicinity of the target SNP.

While the present invention has been described with reference to what are presently considered to be the preferred embodiments, it is to be understood that the invention is not limited to the disclosed embodiments. On the contrary, the invention is intended to cover various modifications and equivalent arrangements included within the spirit and scope of the appended claims. The scope of the following claims is to be accorded the broadest interpretation so as to encompass all such modifications and equivalent structures and functions.

1. A method for amplifying a nucleic acid comprising:
preparing an oligonucleotide being capable of complementarily hybridizing with a specific region of a target nucleic acid containing at least one mutation site, the oligonucleotide having at least one non-complementary sequence being not complementary to any of possible sequences of the at least one mutation site;
subjecting the oligonucleotide to hybridization with the target nucleic acid; and
carrying out a complementary-strand synthesis.

2. The method according to claim 1, wherein the oligonucleotide is allowed to hybridize with the target nucleic acid at temperatures of 45° C. to 55° C.

3. The method according to claim 1, wherein the oligonucleotide is allowed to hybridize with the target nucleic acid at temperatures of 47° C. to 52° C.

4. The method according to claim 1, wherein the non-complementary sequence is positioned at a third to fifteenth base from the 3′ end of the oligonucleotide.

5. The method according to claim 1, wherein the non-complementary sequence is a spacer or a base being not complementary to any of possible sequences of the at least one mutation site.

6. The method according to claim 1, wherein a region of the oligonucleotide capable of hybridizing with the target nucleic acid comprises 17 to 28 bases in length.

7. A method for analyzing a single-nucleotide polymorphism using an amplified product by the method of claim 1.

8. The method according to claim 7, which comprises:
typing a single-nucleotide polymorphism in the target nucleic acid other than the at least one mutation by the analysis of the amount of the amplified product,
wherein a base at the 3′ end or a second base from the 3′ end of the oligonucleotide is so designed as to correspond to the single-nucleotide polymorphism.

9. The method according to claim 8, wherein the typing comprises:
converting pyrophosphate into ATP, the pyrophosphate being generateed as a result of the complementary-strand synthesis;
carrying out a luminous reaction with the use of the resulting ATP and one or more enzymes; and
analyzing the amounts of an amplified product based on the quantity of light emitted as a result of the luminous reaction to thereby type the single-nucleotide polymorphism.

10. The method according to claim 7, which comprises typing a single-nucleotide polymorphism in the target nucleic acid other than the at least one mutation with the use of the amplified product as a template.

11. The method according to claim 10 further comprising:
subjecting an oligonucleotide probe to hybridization with the amplified product, the oligonucleotide probe being so designed as to have a corresponding base at the 3′ end or at a second base from the 3′ end, the base corresponding to the single-nucleotide polymorphism site;
carrying out a complementary-strand synthesis to yield an amplified product; and
typing the single-nucleotide polymorphism by the analysis of the amount of the amplified product.

12. The method according to claim 11, wherein the typing comprises:
converting pyrophosphate into ATP, the pyrophosphate being generateed as a result of the complementary-strand synthesis;
carrying out a luminous reaction with the use of the resulting ATP and one or more enzymes; and
analyzing the amounts of an amplified product based on the quantity of light emitted as a result of the luminous reaction to thereby type the single-nucleotide polymorphism.

13. The method according to claim 10, wherein the oligonucleotide probe has at least one non-complementary sequence being not complementary with any of possible sequences of one or more mutation sites in a region of the target nucleic acid corresponding to the probe other than the single-nucleotide polymorphism.

14. A kit for amplifying a nucleic acid and/or for analyzing a single-nucleotide polymorphism, comprising an oligonucleotide primer or probe being capable of complementarily hybridizing with a specific region of a target nucleic acid containing at least one mutation site,
wherein the oligonucleotide primer or probe has at least one non-complementary sequence being not complementary to any of possible sequences of the at least one mutation site.


SEQUENCE LISTING (9 sequences)

SEQ ID NO: 1 - DNA, 400 bp, Homo sapiens
  Inventor Okano, Kazunori; Nakashima, Yukie
ttctttggcaaccttccaccttyctttctacagaatatttcagtatttctagctcataggttttctaaaataktctctgtaatttattttgaaatggagtttttttatcgtttacagatatgagtaaaattagcctaatcagaatgttagttcctgaaaacattgacacgtaccttatccacatggcaattgagatccttaaacatggtcctgacagyggacttcaaccttcaygtgatgtcaacaaaaggagatgttttcccggttctgaagagatctgttcaagttctaagagaagcaaggaagaagtaggcatcaatactgaggtattaattatatatagaattttcataaagtgtcagtttgttcaatttgcatagcctagtactagaatgctgta

SEQ ID NO: 2 - DNA, 20 bp, Artificial Sequence
  Description of Artificial Sequence Synthetic DNA (primer)
ttccaccttyctttctacag

SEQ ID NO: 3 - DNA, 23 bp, Artificial Sequence
  Description of Artificial Sequence Synthetic DNA (primer)
ctaggctatgcaaattgaacaaa

SEQ ID NO: 4 - DNA, 20 bp, Artificial Sequence
  Description of Artificial Sequence Synthetic DNA (primer of the present invention)
ttccaccttnctttctacag

SEQ ID NO: 5 - DNA, 25 bp, Artificial Sequence
  Description of Artificial Sequence Synthetic DNA (probe)
aacatctccttttgttgacatcacr

SEQ ID NO: 6 - DNA, 530 bp, Homo sapiens
  variation (326): SNP(denoted by SNP93 in [b FIG.9[l )
gaccagtaagttcagagatgcagaggaaaggctgggtccaccctcttaagctcttatatatgattaatacaatcattgcattgatcctcctgtccatgggctgcttgcctgtcctctatcctttggggctggagctccactcacttgacacttctgagccctgaactgccacttcagctgtctccctctggttacaggaagctatgggtcaacccatctgagttcctacctgaacggtttctcacccctgatggtgctatcgacaaggtgttaagtgagaaggtgattatctttggcatgggcaagcggaagtgtatcggtgagamcrttgsccgctgggaggtctttctcttcctggctatcctgctgcaacgggtggaattcagcgtgccactgggcgtgaaggtggacatgacccccatctatgggctaaccatgaagcatgcctgctgtgagcacttccaaatgcagctgcgctcttaggtgcttgagagccctgaggcctagactctgtctacctggtctggttg

SEQ ID NO: 7 - DNA, 24 bp, Artificial Sequence
  Description of Artificial Sequence Synthetic DNA (primer)
gaccagtaagttcagagatgcaga

SEQ ID NO: 8 - DNA, 23 bp, Artificial Sequence
  Description of Artificial Sequence Synthetic DNA (primer)
caaccagaccaggtagacagagt

SEQ ID NO: 9 - DNA, 23 bp, Artificial Sequence
  Description of Artificial Sequence Synthetic DNA (probe of the present invention)
agaaagtcctcccagcggtcaar